(19) United States
(12) Patent Application Publication
(10) Pub. No.: US 2024/0074979 A1
(43) Pub. Date: Mar. 7, 2024

(54) FABRICATION OF TEXTILE-BASED CAPSULES

(71) Applicants: Hadi Dabiryan, Tehran (IR); Saeed Hamze, Tehran (IR)

(72) Inventors: Hadi Dabiryan, Tehran (IR); Saeed Hamze, Tehran (IR)

(73) Assignees: Hadi Dabiryan, Tehran, OT (IR); Saeed Hamze, Tehran, OT (IR); Maryam Khazaei Sani, Noushahar, OT (IR); Amirkabir University of Technology (Tehran Polytechnic) Amirkabir University of Technology (Tehran P, Tehran (IR)

(21) Appl. No.: 18/505,251

(22) Filed: Nov. 9, 2023

Related U.S. Application Data
(63) Continuation-in-part of application No. PCT/IB2021/060782, filed on Nov. 21, 2021.
(60) Provisional application No. 63/186,812, filed on May 11, 2021.

(51) Int. Cl. A61K 9/48

(52) U.S. Cl. CPC A61K 9/4808; A61K 9/4816; A61K 9/4833

(57) ABSTRACT

A method for fabricating a medical braided capsule may include braiding a multi-layer shell onto a powder injection pipe by moving the powder injection pipe linearly up and down in a braiding zone of a braiding machine, discharging a powder from the powder injection pipe into the multi-layer shell, and moving the powder injection pipe out of the multi-layer shell. Discharging the powder from the powder injection pipe into the multi-layer shell and moving the powder injection pipe out of the multi-layer shell are carried out simultaneously.

### CROSS-REFERENCE TO RELATED APPLICATION

This application is a continuation-in-part of PCT/IB2021/060782 filed on Nov. 21, 2021, and entitled “FABRICATION OF TEXTILE-BASED CAPSULES” which claims the benefit of priority from pending U.S. Provisional Patent Application Ser. No. 63/186,812, filed on May 11, 2021, and entitled “A MACHINE FOR PRODUCING A SPECIAL CYLINDRICAL TEXTILE CAPSULATION,” which are both incorporated herein by reference in their entirety.

### TECHNICAL FIELD

The present disclosure generally relates to textile-based capsules and particularly to a system and method for fabricating a textile-based capsule containing powdered and non-powdered pharmaceutical and hygienic materials.

### BACKGROUND

Textiles may be utilized as drug delivery devices in medical and pharmaceutical industries. Specifically, woven carriers may be developed for drug delivery in patients with vaginal or pelvic infections. Powder medicaments or moisture absorbing powders may be encased in a woven or braided outer shell that may allow for absorbing moisture from mucosa to dissolve the encased powdered medicament or absorbent. Consequently, there is a need for developing a system and method that may allow for fabricating braided medical capsules or containers that contain powdered medicaments or superabsorbents. Additionally, there is further a need for developing a soft braided medical capsule that not only prevents damage to the surface of mucosa but also has the necessary consistency for proper placement in a target zone within a user's body and has high capability to absorb moisture from a target zone for either dissolving or releasing a powdered medicament or to absorb and retain moisture.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description and the drawings.

According to one or more exemplary embodiments, the present disclosure is directed to a method for fabricating a medical braided capsule may include braiding a multi-layer shell onto a powder injection pipe by moving the powder injection pipe linearly up and down in a braiding zone of a braiding machine, discharging a powder from the powder injection pipe into the multi-layer shell, and moving the powder injection pipe out of the multi-layer shell. Discharging the powder from the powder injection pipe into the multi-layer shell and moving the powder injection pipe out of the multi-layer shell are carried out simultaneously.

According to one or more exemplary embodiments, the present disclosure is further directed to a braided medical capsule. An exemplary capsule may include a braided multi-layer capsule extended between respective base ends of the braided multi-layer capsule along a longitudinal axis of the braided multi-layer capsule. An exemplary braided multi-layer capsule may include an inner cylindrical layer braided from biocompatible yarns, where the inner cylindrical layer may include a first braided annular wall extended along the longitudinal axis of the braided multi-layer capsule between respective base ends of the braided multi-layer capsule. An exemplary inner cylindrical layer may be configured to encompass a hollow inner volume of the braided multi-layer capsule. and

An exemplary braided multi-layer capsule may further include an outer cylindrical layer continuously braided from the biocompatible yarns over the inner cylindrical layer. An exemplary outer cylindrical layer may include a second braided annular wall extended along and over the first braided annular wall between respective braided base ends of the braided multi-layer capsule. An exemplary second braided annular wall is coaxially extended over an exemplary first braided annular wall. A respective annular base end of the outer cylindrical layer may be continuously attached to a respective annular base end of the inner cylindrical layer. An exemplary medical capsule may further include a powder disposed within the hollow inner volume of the braided multi-layer capsule.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently exemplary embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

FIG. 1A illustrates a sectional front view of a braided medical capsule 100, consistent with one or more exemplary embodiments of the present disclosure;

FIG. 1B illustrates a schematic of braided yarns of a portion of a given layer of a braided multi-layer capsule, consistent with one or more exemplary embodiments of the present disclosure;

FIG. 2A illustrates a flow chart of a method for fabricating a braided medical capsule, consistent with one or more exemplary embodiments of the present disclosure;

FIG. 2B illustrates a flow chart of a method of braiding a multi-layer shell for fabricating a braided medical capsule, consistent with one or more exemplary embodiments of the present disclosure;

FIG. 3 illustrates a block diagram of a system for fabricating a braided medical capsule, consistent with one or more exemplary embodiments of the present disclosure;

FIG. 4A illustrates a front view of an apparatus for fabricating a braided medical capsule, consistent with one or more exemplary embodiments of the present disclosure;

FIG. 4B illustrates a front view of a powder injection assembly, consistent with one or more exemplary embodiments of the present disclosure;

FIGS. 4C-4F illustrate front views of a powder injection pipe moving through a braiding zone, consistent with one or more exemplary embodiments of the present disclosure; and

FIG. 5 illustrates a gripping mechanism of an apparatus for fabricating a braided medical capsule, consistent with one or more exemplary embodiments of the present disclosure.

### DETAILED DESCRIPTION

The novel features which are believed to be characteristic of the present disclosure, as its structure, organization, use and method of operation, together with further objectives and advantages thereof, will be better understood from the following discussion.

The present disclosure is directed to exemplary embodiments of a braided medical capsule that may include a woven shell of threads encasing a powder core. An exemplary powder core of an exemplary braided medical capsule may include a powdered medicament that needs to be released in a target zone of a user's or a patient's body. As used herein, a target zone of a user's or a patient's body may refer to a cavity such as a vaginal canal of a user or patient. An exemplary braided medical capsule may be placed within an exemplary target zone of a patient's body and may release an exemplary powder placed within a core thereof into an exemplary target zone. For example, an exemplary braided medical capsule may be placed internally inside of a user's vaginal canal, where an exemplary braided shell of an exemplary braided medical capsule may become wet, and an exemplary powder core of an exemplary braided medical capsule may be gradually released into a user's vaginal canal due to the braided medical capsule becoming wet and the powder core being dissolved into the moisture.

An exemplary woven or braided shell of an exemplary braided medical capsule may be cylindrical in shape. However, an exemplary braided shell may not be limited to a generally circular perimeter shape and may include other perimeter shapes as long as an exemplary perimeter shape of an exemplary braided shell that may allow for suitably inserting an exemplary braided medical capsule inside a cavity within a user's body. As used herein, suitably inserting may refer to an insertion without imposing any damage to a wall of the cavity or causing discomfort for a patient. An exemplary perimeter shape of an exemplary braided shell may be constant or may vary along a longitudinal axis of an exemplary braided shell. An exemplary braided shell may have a constant diameter or a variable diameter along the longitudinal axis of an exemplary braided shell.

An exemplary braided shell of an exemplary braided medical capsule may include a plurality of interconnected layers that may be continuously braided on top of each other to form a multi-layer braided shell. Each exemplary layer of an exemplary plurality of interconnected layers may be constructed by intertwining a plurality of threads made of cotton by utilizing a braiding machine. Each consecutive layer of an exemplary plurality of interconnected layers may be attached to a respective previous layer of an exemplary plurality of interconnected layers and may be braided on top of a respective previous layer along a longitudinal axis of an exemplary braided shell.

The present disclosure is further directed to a method for fabricating an exemplary braided medical capsule. An exemplary method may allow for braiding an exemplary braided shell of an exemplary braided medical capsule around a powder core by first braiding an exemplary braided shell around a powder injection pipe and then discharging an exemplary powder core into an inner volume of an exemplary braided shell while concurrently pulling an exemplary powder injection pipe out of the inner volume of an exemplary braided shell. To this end, an exemplary braiding machine may be utilized in which an exemplary powder injection pipe may be placed instead of a mandrel of an exemplary braiding machine.

An exemplary powder injection pipe may be placed within an exemplary braiding machine, such that a longitudinal axis of an exemplary powder injection pipe may be aligned with a braiding axis of an exemplary braiding machine. An exemplary powder injection pipe may be placed in the center of a circular platform of an exemplary braiding machine, where a longitudinal axis of an exemplary powder injection pipe may be perpendicular to a plane of an exemplary circular platform. An exemplary circular platform may include a plurality of warp bobbins and a plurality of weft bobbins that may move in opposing serpentine tracks while a plurality of respective warp and weft threads are being unwound and stretched towards an outer surface of an exemplary powder injection pipe at a braiding zone. As used herein, a braiding zone may refer to a zone within an exemplary braiding machine where weft and warp thread are intertwined to form braided patterns on a mandrel, which is replaced herein with a powder injection pipe.

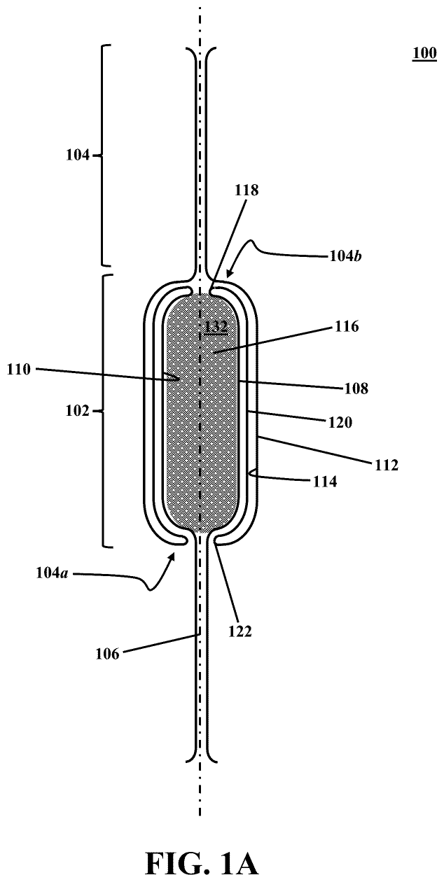

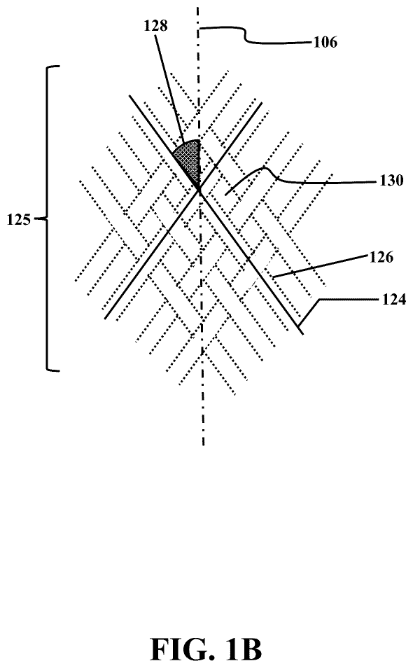

An exemplary powder injection pipe may be translated up and down along a longitudinal axis of an exemplary powder injection pipe. In an exemplary embodiment, translation may refer to a linear movement up and down along a longitudinal axis of an exemplary powder injection pipe. Such exemplary translation of an exemplary powder injection pipe within an exemplary braiding machine may allow for the braids to be constructed on an outer surface of an exemplary powder injection pipe. The aforementioned circular motion of exemplary bobbins of an exemplary braiding machine around an exemplary braiding axis which is superimposed on a longitudinal axis of an exemplary powder injection pipe may allow for rolling up exemplary yarns on an exemplary powder injection pipe, while serpentine or sinusoidal movement of exemplary bobbins may lead to interlacing of the yarns in an exemplary braiding zone. The translation speed of an exemplary powder injection pipe may change the orientation of the yarns on an exemplary powder injection pipe or in other words, the translation speed of an exemplary powder injection pipe may affect the braiding angle. Consequently, an exemplary method may allow for braiding each layer of an exemplary multi-layer shell of an exemplary braided medical capsule with a specific braiding angle, which may be different from previous or consecutive layers to each layer. Exemplary embodiment are described in further detail below with respect to FIGS. 1A-5.

FIG. 1A illustrates a sectional front view of a braided medical capsule 100, consistent with one or more exemplary embodiments of the present disclosure. In an exemplary embodiment, braided medical capsule 100 may include a braided multi-layer capsule 102 that may extend between respective base ends (104a, 104b) of braided multi-layer capsule 102 along a longitudinal axis 106 of braided multi-layer capsule 102. In an exemplary embodiment, braided multi-layer capsule 102 may include an inner layer 108 that may be braided from cotton yarns. In an exemplary embodiment, inner layer 108 may include a first braided annular wall 110 that may extend along longitudinal axis 106 of braided multi-layer capsule 100 between respective base ends (104a, 104b) of braided multi-layer capsule 102. In an exemplary embodiment, inner layer 108 may encompass an inner volume 132 of braided multi-layer capsule 100. In an exemplary embodiment, inner volume 132 of inner layer 108 of braided multi-layer capsule 100 may be filled with a powder 116. In an exemplary embodiment, powder 116 may be a powdered medicament.

In an exemplary embodiment, braided multi-layer capsule 102 may further include an outer layer 112 that may be continuously braided from cotton yarns over inner layer 108. In an exemplary embodiment, outer layer 112 may include a second braided annular wall 114 that may extend coaxially along and over first braided annular wall 110 between respective base ends (104a, 104b) of braided multi-layer capsule 102.

In an exemplary embodiment, braided multi-layer capsule 102 may further include a plurality of intermediate layers that may be continuously braided between inner layer 108 and outer layer 112, where each consecutive braided layer of the plurality of intermediate layers may be coaxially braided around a respective previous layer of the plurality of intermediate layers along longitudinal axis 106 of braided multi-layer capsule 102. For example, braided multi-layer capsule 102 may further include an intermediate layer 120 that may be continuously braided between inner layer 108 and outer layer 112. In an exemplary embodiment, intermediate layer 120 may be coaxially braided around inner layer 108 along longitudinal axis 106 between respective base ends (104a, 104b) of braided multi-layer capsule 102.

In an exemplary embodiment, inner layer 108, outer layer 112, and the plurality of intermediate layers, such as intermediate layer 120 may be braided as a continuous weave. Specifically, in an exemplary embodiment, an exemplary continuous weave may refer to a scenario when a respective base end of each consecutive layer of braided multi-layer capsule 102 may be attached to a respective base end of a previous layer. For example, a respective base end of inner layer 108 may be attached to a respective base end of intermediate layer 120 forming a continuous attachment 118 between inner layer 108 and intermediate layer 120. Similarly, a respective base end of intermediate layer 120 may be attached to a respective base end of outer layer 112 forming a continuous attachment 122 between intermediate layer 120 and outer layer 112. In an exemplary embodiment, continuous attachment 118 between inner layer 108 and intermediate layer 120 is at a first base end 104a of braided multi-layer capsule 102 while continuous attachment 122 between intermediate layer 120 and outer layer 112 is at an opposing second base end 104b. In other words, consecutive layers are continuously braided to their respective previous layer at opposite base ends of braided multi-layer capsule 102.

FIG. 1B illustrates a schematic of braided yarns 125 of a portion of a given layer of braided multi-layer capsule 102, consistent with one or more exemplary embodiments of the present disclosure. As used herein, a braiding angle may refer to an angle 128 between a longitudinal axis 124 of a yarn 126 and longitudinal axis 106 of braided multi-layer capsule 102. In an exemplary embodiment, inner layer 108 may be braided with a first braiding angle, outer layer 112 may be braided with a second braiding angle, and intermediate layer 120 may be braided with a third braiding angle. In an exemplary embodiment, the first braiding angle, the second braiding angle, and the third braiding angle may be similar or equal. For example, the first braiding angle and the second braiding angle may be 30°. In an exemplary embodiment, the first braiding angle, the second braiding angle, and the third braiding angle may be different. In an exemplary embodiment, in case of multiple intermediate layers, a respective braiding angle of a respective layer of the plurality of intermediate layers may be different from a respective braiding angle of a previous layer of the plurality of intermediate layers.

In an exemplary embodiment, each braided layer of braided multi-layer capsule 102 may include diamond-shaped openings or gaps between adjacent threads, such as diamond-shaped gap 130. In an exemplary embodiment, layers of braided multi-layer capsule 102 may be braided with different braiding angles and consequently diamond-shaped openings or gaps within each layer is different in shape and size compared to respective diamond-shaped openings or gaps of adjacent layers. Since exemplary layers of braided multi-layer capsule 102 are braided on top of each other, respective diamond-shaped gaps of exemplary layers are placed on top of each other and due to their different shapes and sizes the openings in the weave of braided multi-layer capsule 102 may not be too dense for moisture and dissolved powder medicament to penetrate through the braids and yet not too open to contain the medicament in its powdered form.

In an exemplary embodiment, braided medical capsule 100 may further include a braided tail 104 that may include an elongated single-layer braided tail continuously attached to a respective base end (104b) of braided multi-layer capsule 102. For example, when braided medical capsule 100 is disposed inside an exemplary vaginal canal, braided tail 104 may be utilized to remove or otherwise pull braided medical capsule 100 out of the exemplary vaginal canal.

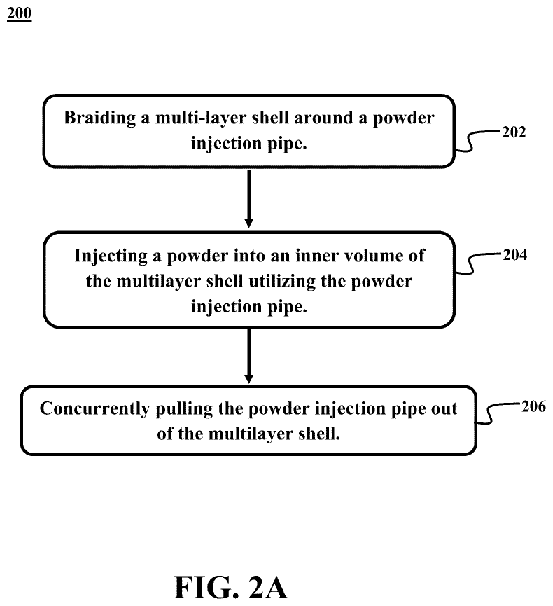

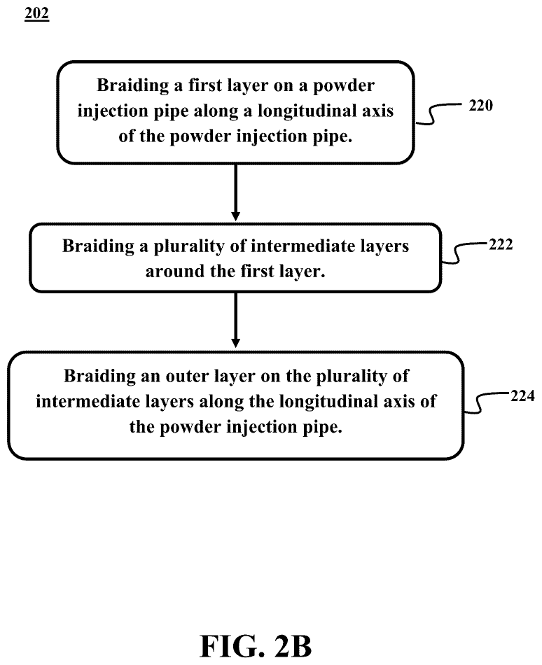

FIG. 2A illustrates a flow chart of a method 200 for fabricating a braided medical capsule, consistent with one or more exemplary embodiments of the present disclosure. In an exemplary embodiment, method 200 may be used to fabricate a braided medical capsule, such as braided medical capsule 100. In an exemplary embodiment, method 200 may include a step 202 of braiding a multi-layer shell around a powder injection pipe, a step 204 of injecting a powder into an inner volume of the multilayer shell utilizing the powder injection pipe, and a step 206 of pulling the powder injection pipe out of the multilayer shell, where steps 204 and 206 are performed simultaneously to maintain the capsule shape of an exemplary braided medical capsule.

As mentioned before, a braided medical capsule, such as braided medical capsule 100 may include a braided multi-layer shell filled with a powdered medicament. In an exemplary embodiment, to braid an exemplary multi-layer shell around an exemplary powder core, an exemplary multi-layer shell may be braided around an exemplary pipe, out of which an exemplary powder is to be discharged. This way, an exemplary pipe may function as a mandrel in an exemplary braiding process and may allow for braiding an exemplary multi-layer shell, an inner volume of which is occupied by an exemplary pipe. After forming an exemplary multi-layer shell, in order to keep a capsule-like shape of the exemplary multi-layer shell, while an exemplary pipe is being pulled out from an inner volume of an exemplary multi-layer shell, an exemplary powder core is discharged into the inner volume of an exemplary multi-layer shell to fill in for an exemplary pipe, which is being pulled out. Consequently, in an exemplary embodiment, step 204 of injecting a powder into an inner volume of the multilayer shell utilizing the powder injection pipe may be carried out simultaneously with step 206 of pulling the powder injection pipe out of the multilayer shell to maintain the shape of an exemplary multilayer shell while an exemplary multilayer shell is being filled with an exemplary powder core. Here, an inner volume of the multilayer shell may refer to a volume encompassed by an innermost layer of the multilayer shell formed immediately on an outer surface of the powder injection pipe. Such volume is occupied by the powder injection pipe but when the powder injection pipe is pulled out of the multilayer shell in step 206. The inner volume once occupied by the powder injection pipe will be filled with the powder.

FIG. 2B illustrates a flow chart of a method of braiding a multi-layer shell (step 202) for fabricating a braided medical capsule, consistent with one or more exemplary embodiments of the present disclosure. In an exemplary embodiment, step 202 of braiding an exemplary multi-layer shell around an exemplary powder injection pipe may include a step 220 of braiding a first layer on a powder injection pipe along a longitudinal axis of the powder injection pipe, a step 222 of braiding a plurality of intermediate layers around the first layer, where each consecutive intermediate layer of the plurality of intermediate layers may be coaxially braided around a respective previous intermediate layer of the plurality of intermediate layers along the longitudinal axis of the powder injection pipe, and a step 224 of braiding an outer layer on the plurality of intermediate layers along the longitudinal axis of the powder injection pipe.

In an exemplary embodiment, step 220 of braiding the first layer on the powder injection pipe along the longitudinal axis of the powder injection pipe may involve moving the powder injection pipe through the braiding zone of a braiding machine in a first direction along the longitudinal axis of the powder injection pipe. A linear translational motion of the powder injection pipe along the first direction may allow for braids to be constructed on at least a portion of the powder injection pipe. After braiding the first layer on the outer surface of the powder injection pipe, method 200 may proceed to step 222 of braiding the plurality of intermediate layers around the first layer. In order to braid a first intermediate layer of the plurality of intermediate layers on the first layer, the braiding process may be continuously carried out however this time the powder injection pipe may be translated through the braiding zone in an opposing second direction along the longitudinal axis of the powder injection pipe, which allows for continuously braiding the first intermediate layer of the plurality of intermediate layers on top of the first layer along the longitudinal axis of the powder injection pipe. Similarly, respective consecutive intermediate layers of the plurality of intermediate layers may be braided on top of ach other by changing the direction of translational movement of the powder injection pipe for braiding each consecutive intermediate layer.

After braiding the plurality of intermediate layers, method 200 may proceed to step 224 of braiding an outer layer on the plurality of intermediate layers along the longitudinal axis of the powder injection pipe. Similarly, in an exemplary embodiment, responsive to moving the powder injection pipe in a first direction through the braiding zone to braid the last intermediate layer of the plurality of intermediate layers, in order to braid the outer layer, the powder injection pipe may be moved in an opposing second direction such that braids may be continuously formed on top of the last intermediate layer of the plurality of intermediate layers. Such continuous braiding of the first layer, the plurality of intermediate layers, and the outer layer may allow for having a multi-layer braided shell, where all layers form a continuous weave and are interconnected, instead of being a plurality of discrete layers disposed on top of each other.

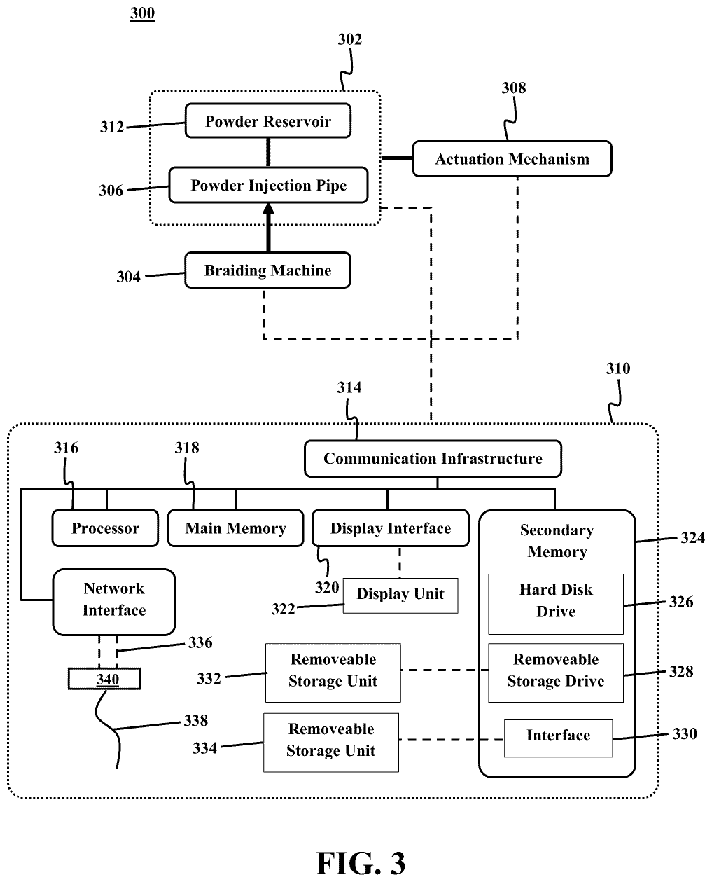

FIG. 3 illustrates a block diagram of a system 300 for fabricating a braided medical capsule, consistent with one or more exemplary embodiments of the present disclosure. In an exemplary embodiment, system 300 may be configured to perform a method similar to method 200 to fabricate a braided medical capsule similar to braided medical capsule 100, that is, all the elements of system 300 may be utilized to perform exemplary methods similar to exemplary method 200

In an exemplary embodiment, system 300 may include a powder injection assembly 302, a braiding machine 304 configured to braid a multi-layer braided shell around a powder injection pipe 306 of powder injection assembly 302, an actuation mechanism 308 configured to translate powder injection pipe 306 along a longitudinal axis of powder injection pipe 306, and a control system 310 in signal communication with each relevant component and mechanism of system 300 responsible for automatically fabricating a braided medical capsule, as will be discussed.

In an exemplary embodiment, powder injection assembly 302 which may be utilized for injecting a powder into a braided multilayer capsule, may include a powder reservoir 312 that may be connected to powder injection pipe 306. In an exemplary embodiment, powder reservoir 312 may be a hopper equipped with an actuator, such as a screw that may deliver a powder from powder reservoir 312 into powder injection pipe 306 on demand. For example, a screw may be mounted within powder reservoir 312 with an axis of rotation of the screw being along the longitudinal axis of powder injection pipe 306. A rotational movement of the screw may force the powder to move toward powder injection pipe 306 and eventually be discharged from powder injection pipe 306. In an exemplary embodiment, powder reservoir 312 may further include baffles and mixing blades disposed within powder reservoir to prevent the powder from agglomerating.

In an exemplary embodiment, control system 310 may be in signal communication with powder injection assembly 302 and may urge powder injection assembly 302 to discharge the powder from powder injection pipe 306. For example, an exemplary screw within powder reservoir 312 may be actuated by an electric motor. An exemplary driver of an exemplary electric motor may be in signal communication with control system 310, where control system 310 may control injection of the powder by turning the electric motor on and off by sending a control signal to the motor. In an exemplary embodiment, the discharge rate of the powder from powder injection pipe 306 depends on the rotational speed of the screw and the pitch of the screw. As used herein, the pitch of the screw refers to the distance or spacing between adjacent threads of the screw.

In an exemplary embodiment, braiding machine 304 may be coupled with powder injection assembly 302 such that the longitudinal axis of powder injection pipe 306 may be superimposed on a braiding axis of braiding machine 304. In an exemplary embodiment, braiding machine 304 may be configured to braid a multi-layer shell of individual threads around an outer surface of powder injection pipe 306, that is, with all components of braiding machine 304 functioning to perform a method similar to steps 202 to 206 of method 200, a multi-layer shell of individual threads may be braided. In an exemplary embodiment, powder injection pipe 306 may be placed in the center of braiding machine 304 along the braiding axis of braiding machine 304. Furthermore, powder injection pipe 306 may be translated along the braiding axis during the braiding process to allow for multi-layer braids to be constructed on at least a portion of the outer surface of powder injection pipe 306. To this end, in an exemplary embodiment, actuation mechanism 308 may be coupled to powder injection assembly 302 and may be configured to drive a translational movement of powder injection assembly 302 along the braiding axis of braiding machine 304. In an exemplary embodiment, actuation mechanism 308 may include a linear actuator that may be in signal communication with control system 310, where control system 310 may be configured to urge actuation mechanism 308 to actuate a linear motion of powder injection assembly 302 along the braiding axis of braiding machine 304. In practice, powder injection assembly 302 may be configured to inject a powder stored within powder reservoir 312 into an exemplary multi-layer shell braided around powder injection pipe 306 in response to braiding machine 304 braiding the last layer of an exemplary multi-layer shell around powder injection pipe 306.

In an exemplary embodiment, control system 310 may be implemented as a computer system, in which an embodiment of the present disclosure, or portions thereof, may be implemented as computer-readable code, consistent with exemplary embodiments of the present disclosure. For example, control system 310 may be implemented in a computer system using hardware, software, firmware, tangible computer-readable media having instructions stored thereon, or a combination thereof and may be implemented in one or more computer systems or other processing systems.

If programmable logic is used, such logic may be executed on a commercially available processing platform or a special purpose device. An embodiment of the disclosed subject matter may be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this control system 310. After reading this description, it will become apparent to a person skilled in the relevant art how to implement the invention using other computer systems and/or computer architectures. Although operations may be described as a sequential process, some of the operations may be performed in parallel, concurrently, and/or in a distributed environment, and with program code stored locally or remotely for access by single or multi-processor machines. Also, in some embodiments, the order of operations may be rearranged without departing from the spirit of the disclosed subject matter.

Processor device 316 may be a special purpose or a general-purpose processor device. As will be appreciated by persons skilled in the relevant art, processor device 316 may also be a single processor in a multi-core/multiprocessor system, such system operating alone, or in a cluster of computing devices operating in a cluster or server farm. Processor device 316 may be connected to a communication infrastructure 314, for example, a bus, message queue, network, or multi-core message-passing scheme.

In an exemplary embodiment, control system 310 may include a display interface 320, for example, a video connector, to transfer data to a display unit 322, for example, a monitor. Control system 310 may also include a main memory 318, for example, random access memory (RAM), and may also include a secondary memory 324. Secondary memory 324 may include, for example, a hard disk drive 326, and a removable storage drive 328. Removable storage drive 328 may include a floppy disk drive, a magnetic tape drive, an optical disk drive, a flash memory, or the like. Removable storage drive 328 may read from and/or write to a removable storage unit 332 in a well-known manner. Removable storage unit 332 may include a floppy disk, a magnetic tape, an optical disk, etc., which may be read by and written to by removable storage drive 328. As will be appreciated by persons skilled in the relevant art, removable storage unit 332 may include a computer-usable storage medium having stored therein computer software and/or data.

In alternative implementations, secondary memory 324 may include other similar means for allowing computer programs or other instructions to be loaded into control system 310. Such means may include, for example, a removable storage unit 334 and an interface 330. Examples of such means may include a program cartridge and cartridge interface (such as that found in video game devices), a removable memory chip (such as an EPROM, or PROM) and associated socket, and other removable storage units 334 and interfaces 330 which allow software and data to be transferred from removable storage unit 334 to control system 310.

Control system 310 may also include a communications interface 340. Communications interface 340 allows software and data to be transferred between control system 310 and external devices. Communications interface 340 may include a modem, a network interface (such as an Ethernet card), a communications port, a PCMCIA slot, and card, or the like. Software and data transferred via communications interface 340 may be in the form of signals, which may be electronic, electromagnetic, optical, or other signals capable of being received by communications interface 340. These signals may be provided to communications interface 340 via a communications path 338. Communications path 338 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 332, removable storage unit 334, and a hard disk installed in hard disk drive 326. Computer program medium and computer-usable medium may also refer to memories, such as main memory 318 and secondary memory 324, which may be memory semiconductors (e.g. DRAMs, etc.).

Computer programs (also called computer control logic) are stored in main memory 318 and/or secondary memory 324. Computer programs may also be received via communications interface 340. Such computer programs, when executed, enable control system 310 to implement different embodiments of the present disclosure as discussed herein. In particular, the computer programs, when executed, enable processor device 316 to implement the processes of the present disclosure, such as the operations in method 100. Accordingly, such computer programs represent controllers of control system 310. Where an exemplary embodiment of method 100 is implemented using software, the software may be stored in a computer program product and loaded into control system 310 using removable storage drive 328, interface 330, and hard disk drive 326, or communications interface 340.

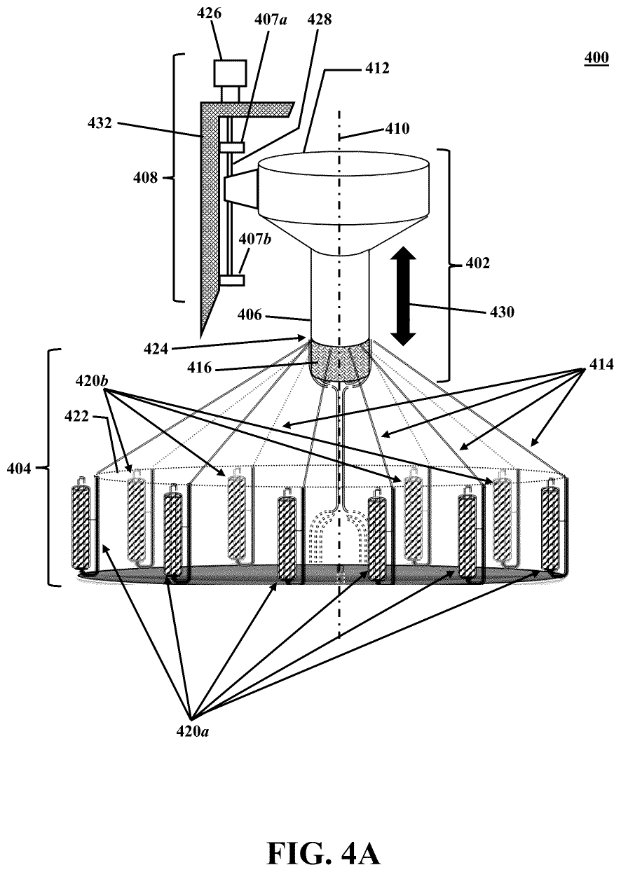

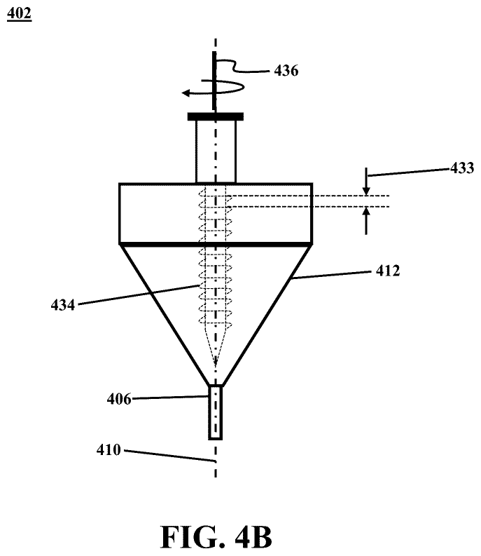

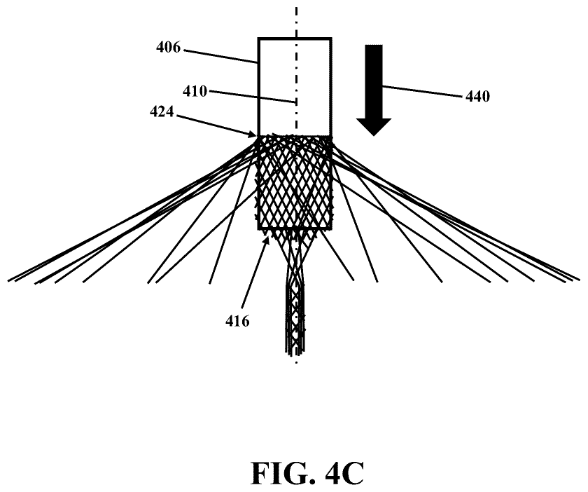

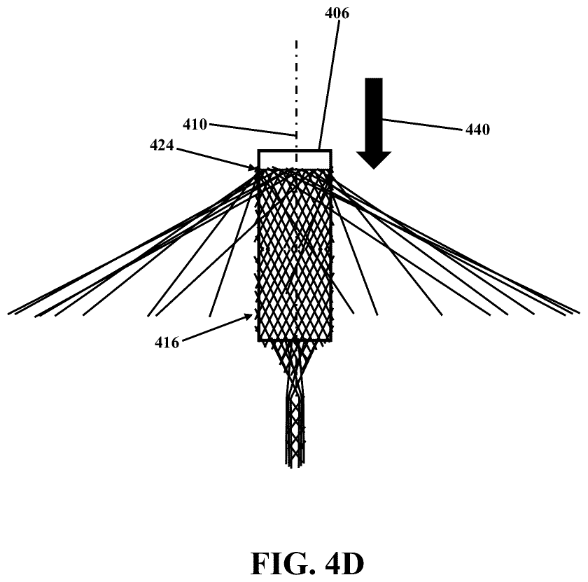

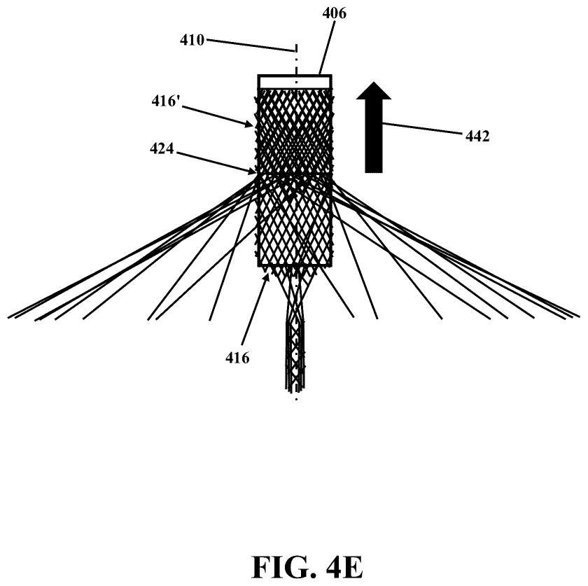

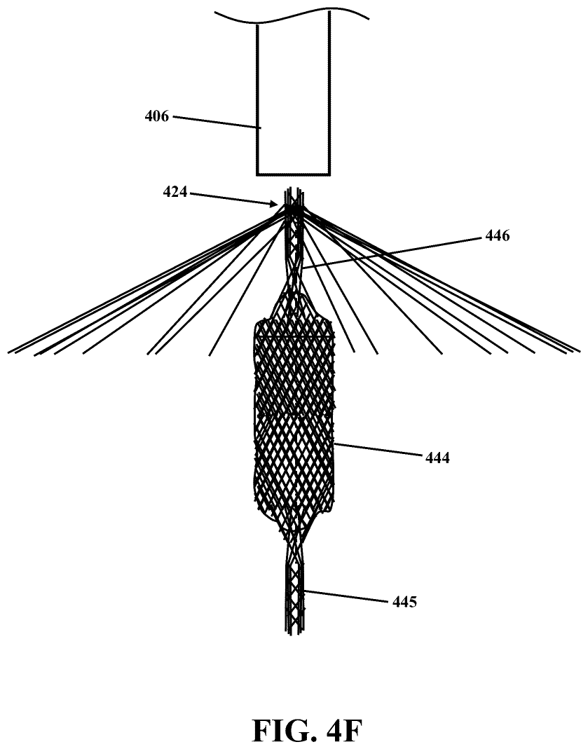

FIG. 4A illustrates a front view of an apparatus 400 for fabricating a braided medical capsule, consistent with one or more exemplary embodiments of the present disclosure. In an exemplary embodiment, apparatus 400 may be structurally similar to system 300 and may be configured to perform a method similar to method 200 to fabricate a braided medical capsule similar to braided medical capsule 100.

In an exemplary embodiment, apparatus 400 may include a powder injection assembly 402 similar to powder injection assembly 302, a braiding machine 404 similar to braiding machine 304 that may braid a multi-layer braided shell around a powder injection pipe 406 of powder injection assembly 402, and an actuation mechanism 408 similar to actuation mechanism 308 that may be configured to translate powder injection pipe 406 along a longitudinal axis 410 of powder injection pipe 406.

FIG. 4B illustrates a front view of powder injection assembly 402, consistent with one or more exemplary embodiments of the present disclosure. In an exemplary embodiment, powder injection assembly 402 may include a powder reservoir 412 similar to powder reservoir 312 that may be connected to powder injection pipe 406. In an exemplary embodiment, since medical substances are t be handles by utilizing powder injection assembly 402, all components of powder injection assembly 402 in contact with medical substances, such as the powder medicament, should be made of medical grade stainless steel. For example, powder reservoir 412 and powder injection pipe 406 may be made of stainless steel type 316.

In an exemplary embodiment, powder reservoir 412 may be a hopper equipped with a powder injection mechanism, such as a screw 434 that may deliver a powder from powder reservoir 412 into powder injection pipe 406 on demand. For example, screw 434 may be mounted within powder reservoir 412 with an axis of rotation 436 of screw 434 being along longitudinal axis 410 of powder injection pipe 406. In an exemplary embodiment, a rotary actuator such as an electric motor 438 may be utilized for driving a rotational movement of screw 434 about axis of rotation 436. Such rotational movement of screw 434 may force the powder to move toward powder injection pipe 406 and eventually be discharged out of powder injection pipe 406. In an exemplary embodiment, as mentioned before, the discharge rate of the powder from powder injection pipe 306 may depend on the rotational speed of screw 434 and pitch 433 of screw 434. In an exemplary embodiment, pitch 433 is the distance or spacing between two adjacent threads of screw 434.

In an exemplary embodiment, braiding machine 404 may include a plurality of threads 414 that may be intertwined to form braided weave patterns 416 on an outer surface of powder injection pipe 406, where braided weave patterns 416 may eventually form an exemplary braided shell of an exemplary braided medical capsule similar to braided medical capsule 100. In an exemplary embodiment, plurality of threads 414 may be formed from cotton. In an exemplary embodiment, braiding machine 404 may further include a circular platform 418, on which a plurality of bobbins (420a, 420b) may be mounted. In an exemplary embodiment, circular platform 418 may be configured to rotate plurality of bobbins (420a, 420b) around powder injection pipe 406. In an exemplary embodiment, each bobbin of plurality of bobbins (420a, 420b) may include a respective thread of plurality of threads 414 wound around each bobbin of plurality of bobbins (420a, 420b) for use in the braiding process. An exemplary thread of each bobbin of plurality of bobbins (420a, 420b) may be redirected by a rim 422 radially inwardly towards a braiding zone 424. In an exemplary embodiment, each unwinding thread of plurality of threads 414 may be tensioned over rim 422 between a respective bobbin of plurality of bobbins (420a, 420b) and braiding zone 424. In other words, each thread of plurality of threads 414 may extend away from rim 422 in a radial inward direction toward powder injection pipe 406, where the outer surface of powder injection pipe 406 may be instantaneously covered by braided weave patterns 416.

In an exemplary embodiment, plurality of bobbins (420a, 420b) may include a first set of bobbins 420a that may be configured to orbit around rim 422 in a first orbiting direction and a second set of bobbins 420b that may be configured to orbit around rim 422 in a second orbiting direction arranged opposite the first orbiting direction. In an exemplary embodiment, circular platform 418 may include a first track extending around rim 422 and a second track similarly extending around rim 422. In an exemplary embodiment, the first track and the second track which are not illustrated for simplicity may be of a serpentine shape and first set of bobbins 420a may be configured to traverse the first track while second set of bobbins 420b may traverse the second track. In an exemplary embodiment, first set of bobbins 420a and second set of bobbins 420b may move along prescribed tracks, namely, the first and second tracks in opposing orbiting directions while maintaining the tension of each respective thread of plurality of threads 414 when extending over and contacting rim 422.

In an exemplary embodiment, the first and second tracks may be disposed radially outwardly of rim 422 and cross over each other in the radial direction of rim 422 to facilitate first set of bobbins 420a repeatedly passing radially inwardly and then radially outwardly of second set of bobbins 420b when first set of bobbins 420a and second set of bobbins 420b are orbited around rim 422 in the opposing orbiting directions. In an exemplary embodiment, such in-and-out motion of first set of bobbins 420a and second set of bobbins 420b may allow for forming braided weave patterns 416 where oppositely sloped threads of plurality of threads 414 may alternatingly pass in front and behind each other.

FIGS. 4C-4F illustrate front views of powder injection pipe 406 moving through braiding zone 424, consistent with one or more exemplary embodiments of the present disclosure. In an exemplary embodiment, powder injection pipe 406 may be moved vertically along longitudinal axis 410 of powder injection pipe 406 to allow the outer surface of powder injection pipe 406 to be positioned in braiding zone 424, where braided weave patterns 416 may be gradually braided onto the outer surface of powder injection pipe 410. For example, referring to FIG. 4C, powder injection pipe 406 may be moved vertically along longitudinal axis 410 in a direction shown by arrow 440 and a first layer of braided weave patterns 416 may be gradually braided onto the outer surface of powder injection pipe 406. Referring to FIG. 4D, translational movement of powder injection pipe 406 through braiding zone 424 may be continued to the point where a desired portion of powder injection pipe 406 may be covered by a first layer of braided weave patterns 416. Then, powder injection pipe 406 may be translated along longitudinal axis 410 in an opposing direction to allow for braiding a second consecutive layer of braided weave patterns onto the outer surface of powder injection pipe 406 over the first layer of braided weave patterns 416. For example, referring to FIG. 4E, powder injection pipe 406 may be translated along longitudinal axis 410 in a direction shown by arrow 440 and a consecutive layer of braided weave patterns 416′ may be gradually braided onto the outer surface of powder injection pipe 406 over the first layer of braided weave patterns 416. In an exemplary embodiment, consecutive layer of braided weave patterns 416′ may be different from the first layer of braided weave patterns 416 in that braiding angle of braided weave patterns 416′ may be different from braiding angle of braided weave patterns 416. In an exemplary embodiment, powder injection pipe 406 may be translated along longitudinal axis 410 with different linear translation speeds for each individual layer of braided weave patterns and this way each individual layer of braided weave patterns may be braided with a different braiding angle. For example, powder injection pipe 406 may be translated along longitudinal axis 410 in the direction shown by arrow 440 with a first translational speed and powder injection pipe 406 may be translated along longitudinal axis 410 in the direction shown by arrow 440 with a second translational speed. In an exemplary embodiment, the first translational speed and the second translational speed may be different.

In an exemplary embodiment, actuation mechanism 408 may be coupled to powder injection assembly 402 and may be configured to translate powder injection assembly 402 along longitudinal axis 410 of powder injection pipe 406.

In an exemplary embodiment, each consecutive layer may be braided on a respective previous layer on the outer surface of powder injection pipe 406 by changing the direction of translational movement of powder injection pipe 406, consequently, a base end of each layer is continuously braided with a base end of the next layer, as if all the layers are one continuous weave that is folded layer by layer.

In an exemplary embodiment, such translational movement of powder injection pipe 406 may be actuated by utilizing actuation mechanism 408. In an exemplary embodiment, actuation mechanism 408 may include a motor 426 that may be coupled to a mechanical linear actuator, such as a ball screw 428. In an exemplary embodiment, powder injection assembly 402 may be moveably coupled to ball screw 428, where ball screw 428 may translate rotational motion of motor 426 to linear motion of powder injection assembly 402. In an exemplary embodiment, actuation mechanism 408 may be mounted on a frame 432 and may be configured to translate powder injection assembly 402 along longitudinal axis 410 in directions shown by arrow 430 relative to frame 432. Specifically, a rotational movement of motor 426 in a first rotational direction may cause powder injection assembly 402 to be translated in a first direction while a rotational movement of motor 426 in an opposing second rotational direction may cause powder injection assembly 402 to be translated in an opposing second direction. Consequently, upward or downward motion of powder injection pipe 406 along longitudinal axis 410 may be controlled by manipulating the direction of rotational motion of motor 426 and the speed at which powder injection pipe 406 may be translated along longitudinal axis 410 may be controlled by manipulating the rotational speed of motor 426.

In an exemplary embodiment, motor 426 may include a servo motor in signal communication with a control system similar to control system 310. In an exemplary embodiment, an exemplary control system similar to control system 310 may be configured to control the speed and direction of the rotational movement of motor 426. In an exemplary embodiment, actuation mechanism 408 may further include sensors, such as a coupled of microswitches (407a, 407b) that may be coupled with ball screw 428. In an exemplary embodiment, microswitches (407a, 407b) may be mounted at respective extreme ends of a translational path defined by ball screw 428. In an exemplary embodiment, signal output of microswitches (407a, 407b) may be utilized by an exemplary control unit to determine the position of powder injection pipe 406 along the translational path defined by ball screw 428.

In an exemplary embodiment, after braiding a braided multi-layer shell 444 around the outer surface of powder injection pipe 406 with a desired number of layers, it is time for the powder injection pipe 406 to be replaced by the powder core. To this end, in an exemplary embodiment, actuation mechanism 408 may be configured to translate powder injection pipe 406 out of braided multi-layer shell 444 and away from braiding zone 424, as for example illustrated in FIG. 4F. In order to maintain the capsule shape of braided multi-layer shell 444, pulling powder injection pipe 406 out of braided multi-layer shell 444 may be carried out simultaneously with discharging the powder core from powder injection pipe 406 into braided multi-layer shell 444. To this end, powder injection assembly 402 may be configured to discharge the powder core into braided multi-layer shell 444 via powder injection pipe 406 while powder injection pipe 406 is being pulled out of braided multi-layer shell 444. Such configuration of powder injection assembly 402 may allow for filling braided multi-layer shell 444 with a controlled amount of the powder core with a controlled density.

Referring to FIG. 4F, in an exemplary embodiment, after powder injection pipe 406 is pulled out of braiding zone 424 and braided multi-layer shell 444 is filled with the powder core, the braiding machine continues to braid a braided tail 446 of a next braided multi-layer medical capsule. The next multi-layer medical capsule may be constructed by moving powder injection pipe 406 down into braiding zone 424 after braided tail 446 is braided to a desired length. In an exemplary embodiment, braided multi-layer shell 444 filled with the powder core and a braided tail 445 attached to a lower end of braided multi-layer shell 444 together form a braided medical capsule similar to braided medical capsule 100.

In an exemplary embodiment, an exemplary apparatus or device similar to apparatus 400 for fabricating a braided medical capsule may allow for continuously fabrication a plurality of braided medical capsules as was discussed in the previous paragraph. Exemplary braided medical capsules may be braided and filled with the powder core continuously one after the other by utilizing apparatus 400 to implement method 200 of fabricating braided medical capsules. For example, step 220 of braiding the first layer on the powder injection pipe along the longitudinal axis of the powder injection pipe may include moving powder injection pipe 406 through braiding zone 424 of braiding machine 404 along longitudinal axis 410 in the direction of arrow 440. For example, step 222 of braiding the plurality of intermediate layers around the first layer and step 224 of braiding the outer layer on the plurality of intermediate layers along the longitudinal axis of the powder injection pipe may be carried out by alternately moving powder injection pipe 406 through braiding zone 424 of braiding machine 404 along longitudinal axis 410 up and down as shown by arrows (440, 442) until a desired number of layers are braided onto the outer surface of powder injection pipe 406.

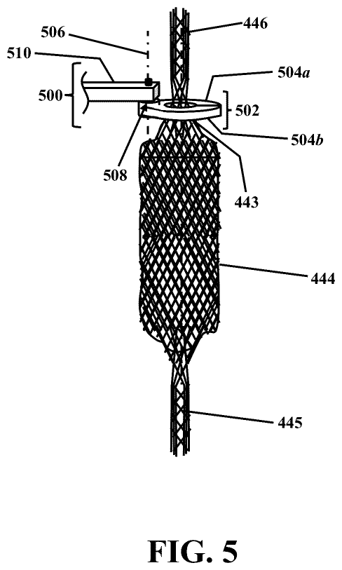

FIG. 5 illustrates a gripping mechanism 500 of an apparatus for fabricating a braided medical capsule, consistent with one or more exemplary embodiments of the present disclosure. In an exemplary embodiment, when a multilayer shell with a desired number of layers is braided on an exemplary powder injection pipe and an exemplary powder injection pipe has been pulled out of an exemplary multilayer shell, a gripping mechanism may be utilized for both closing an exemplary base end of an exemplary multilayer shell and gripping an exemplary multilayer shell when an exemplary powder injection pipe is being pulled away from an exemplary braiding zone. For example, apparatus 400 may further include a gripping mechanism, such as gripping mechanism 500 that may be configured to grip a based end 443 of braided multi-layer shell 444 responsive to an exemplary powder injection pipe having been pulled out of braided multi-layer shell 444 and an exemplary powder core having been injected into braided multi-layer shell 444.

In an exemplary embodiment, gripping mechanism 500 may include a gripping jaw 502 that may be actuated between an open and a close position. In an exemplary embodiment, gripping jaw 502 may include two fingers, namely a first finger 504a and a second finger 504b that may be coupled to each other by utilizing a pivot joint 508 on a distal end of a support arm 510. In an exemplary embodiment, first finger 504a and second finger 504b may be urged to pivot on pivot joint 508 about a pivot axis 506 between an open state where first finger 504a and second finger 504b are pivoted away from each other and a close state where first finger 504a and second finger 504b are pivoted toward each other.

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word “substantially” when used with an adjective or adverb is intended to enhance the scope of the particular characteristic, e.g., substantially planar is intended to mean planar, nearly planar and/or exhibiting characteristics associated with a planar element. Further use of relative terms such as “vertical”, “horizontal”, “up”, “down”, and “side-to-side” are used in a relative sense to the normal orientation of the apparatus.

What is claimed is:

1. A braided medical capsule, comprising:
a braided multi-layer capsule comprising:
an inner cylindrical layer braided from biocompatible yarns with a first braiding angle, the inner cylindrical layer comprising a first braided annular wall encompassing a hollow inner volume of the braided multi-layer capsule, the inner cylindrical layer extended along a longitudinal axis of the braided multi-layer capsule between respective base ends of the braided multi-layer capsule; and
an outer cylindrical layer continuously braided from the biocompatible yarns with a second braiding angle over the inner cylindrical layer, the second braiding angle and the first braiding angle being equal to 30°, the outer cylindrical layer comprising a second braided annular wall extended coaxially along and over the first braided annular wall between respective braided base ends of the braided multi-layer capsule, a respective annular base end of the outer cylindrical layer attached to a respective annular base end of the inner cylindrical layer; and
a powder disposed within the hollow inner volume of the braided multi-layer capsule, the powder comprising a powder medicament;
a plurality of intermediate cylindrical layers continuously braided between the inner cylindrical layer and the outer cylindrical layer, each consecutive braided cylindrical layer of the plurality of intermediate cylindrical layers coaxially braided around a respective previous cylindrical layer of the plurality of intermediate cylindrical layers along the longitudinal axis of the braided multi-layer capsule, a respective base end of each consecutive cylindrical layer of the plurality of intermediate cylindrical layers attached to a respective base end of a previous cylindrical layer of the plurality of intermediate cylindrical layers, the inner cylindrical layer, the outer cylindrical layer, and the plurality of intermediate cylindrical layers forming a continuous weave, a respective braiding angle of a respective cylindrical layer of the plurality of intermediate cylindrical layers being different from a respective braiding angle of a previous cylindrical layer of the plurality of intermediate cylindrical layers, the braiding angle comprising an angle between respective yams of a respective cylindrical layer of the plurality of intermediate cylindrical layers and the longitudinal axis of the braided multi-layer capsule; and
a braided tail, the braided tail comprising an elongated single-layer braided tail continuously attached to a respective base end of the braided multi-layer capsule.

2. A braided medical capsule, comprising:
a braided multi-layer capsule comprising:
an inner cylindrical layer braided from biocompatible yarns, the inner cylindrical layer comprising a first braided annular wall encompassing a hollow inner volume of the braided multi-layer capsule, the inner cylindrical layer extended along a longitudinal axis of the braided multi-layer capsule between respective base ends of the braided multi-layer capsule; and
an outer cylindrical layer continuously braided from the biocompatible yarns over the inner cylindrical layer, the outer cylindrical layer comprising a second braided annular wall extended coaxially along and over the first braided annular wall between respective braided base ends of the braided multi-layer capsule, a respective annular base end of the outer cylindrical layer attached to a respective annular base end of the inner cylindrical layer; and
a powder disposed within the hollow inner volume of the braided multi-layer capsule.

3. The braided medical capsule of claim 2, further comprising a plurality of intermediate cylindrical layers continuously braided between the inner cylindrical layer and the outer cylindrical layer, each consecutive braided cylindrical layer of the plurality of intermediate cylindrical layers coaxially braided around a respective previous cylindrical layer of the plurality of intermediate cylindrical layers along the longitudinal axis of the braided multi-layer capsule, a respective base end of each consecutive cylindrical layer of the plurality of intermediate cylindrical layers attached to a respective base end of a previous cylindrical layer of the plurality of intermediate cylindrical layers.

4. The braided medical capsule of claim 3, wherein the inner cylindrical layer, the outer cylindrical layer, and the plurality of intermediate cylindrical layers form a continuous weave.

5. The braided medical capsule of claim 4, wherein a respective braiding angle of a respective cylindrical layer of the plurality of intermediate cylindrical layers is different from a respective braiding angle of a previous cylindrical layer of the plurality of intermediate cylindrical layers, the braiding angle comprising an angle between respective yarns of a respective cylindrical layer of the plurality of intermediate cylindrical layers and the longitudinal axis of the braided multi-layer capsule.

6. The braided medical capsule of claim 2, wherein the inner cylindrical layer is braided from the biocompatible yarns with a first braiding angle, and wherein the outer cylindrical layer is braided from the biocompatible yarns with a second braiding angle.

7. The braided medical capsule of claim 6, wherein the first braiding angle is equal to the second braiding angle.

8. The braided medical capsule of claim 7, wherein the first braiding angle is not equal to the second braiding angle.

9. The braided medical capsule of claim 2, wherein the powder comprises a powder medicament.

10. The braided medical capsule of claim 2, further comprising a braided tail, the braided tail comprising an elongated single-layer braided tail continuously attached to a respective base end of the braided multi-layer capsule.

11. A method for fabricating a medical braided capsule, the method comprising:
braiding a multi-layer shell onto a powder injection pipe by moving the powder injection pipe linearly up and down in a braiding zone of a braiding machine;
discharging a powder from the powder injection pipe into the multi-layer shell; and
moving the powder injection pipe out of the multi-layer shell,
wherein discharging the powder from the powder injection pipe into the multi-layer shell and moving the powder injection pipe out of the multi-layer shell are carried out simultaneously.

12. The method of claim 10, wherein braiding the multi-layer shell onto the powder injection pipe comprises braiding the multi-layer shell onto at least a portion of an outer surface of the powder injection pipe.

13. The method of claim 10, wherein braiding the multi-layer shell onto the powder injection pipe comprises braiding a plurality of layers of the multi-layer shell onto the powder injection pipe, each layer of the plurality of layers braided on top of a respective previous layer of the plurality of layers along a longitudinal axis of the powder injection pipe, a respective base end of each layer of the plurality of layers continuously attached to a respective base end of a previous layer of the plurality of layers.

14. The method of claim 12, wherein braiding the plurality of layers onto the powder injection pipe comprises moving the injection pipe along the longitudinal axis of the powder injection pipe through the braiding zone of the braiding machine.

15. The method of claim 13, wherein braiding the plurality of layers onto the powder injection pipe further comprises braiding each layer of the plurality of layers with a respective braiding angle, the respective braiding angle of each layer of the plurality of layers different from a respective braiding angle of a respective previous layer of the plurality of layers.

16. The method of claim 14, wherein braiding each layer of the plurality of layers with a respective braiding angle comprises braiding each layer of the plurality of layers by moving the powder injection pipe along the longitudinal axis of the powder injection pipe with a respective translational speed.

17. The method of claim 15, wherein braiding the multi-layer shell onto the powder injection pipe comprises braiding a multi-layer shell of cotton yarns onto the powder injection pipe.

18. The method of claim 16, wherein discharging the powder from the powder injection pipe into the multi-layer shell comprises discharging a powdered medicament from the powder injection pipe into the multi-layer shell.

19. The method of claim 10, wherein discharging the powder from the powder injection pipe into the multi-layer shell is carried out in response to the multi-layer shell having been braided onto the powder injection pipe.
